(19) United States
(12) Patent Application Publication
(10) Pub. No.: US 2025/0099527 A1
(43) Pub. Date: Mar. 27, 2025

(54) Potentiated Ashwagandha roots compound with Improved Efficacy

(71) Applicant: Bright Lifecare Pvt. Ltd, Gurgaon (IN)

(72) Inventors: Anupam Trehan, Gurgaon (IN); Manoj Kumar Verma, Gurgaon (IN); Anurag Gupta, Gurgaon (IN); Rachna Anand, Gurgaon (IN); Gopal Taneja, Gurgaon (IN); Sameer Maheshwari, Gurgaon (IN)

(73) Assignee: Bright Lifecare Pvt. Ltd, Gurgaon (IN)

(21) Appl. No.: 18/893,523

(22) Filed: Sep. 23, 2024

(30) Foreign Application Priority Data
Sep. 26, 2023 (IN) 202311064713

(51) Int. Cl. A61K 36/81; A61K 35/20; B01D 11/02

(52) U.S. Cl. CPC A61K 36/81; A61K 35/20; B01D 11/0288; A61K 2236/331; A61K 2236/39; A61K 2236/51

(57) ABSTRACT

The present invention relates to ashwagandha root extract that potentiates the complete spectrum of natural active principles present in the Ashwagandha root. The composition is prepared by a process that uses no organic solvent media to extract active principles. The compositions are further potentiated with milk or with biproducts of milk like whey, milk solids, curd etc.

### TECHNICAL FIELD OF THE INVENTION

The present invention relates to ashwagandha root extract that potentiates the complete spectrum of natural active principles present in the Ashwagandha root. The composition is prepared by a process that uses no organic solvent media to extract active principles. The compositions are further potentiated with milk or with biproducts of milk like whey, milk solids, curd etc.

### BACKGROUND OF THE INVENTION

Ayurvedic drugs can also be described as the natural herb/herbo-mineral/herbo-metallic drugs of Indian System of Medicine, are drugs composed of natural substances which have constituents that supplement health or have medicinal activities.

In ayurveda, one medicinal plant of repute importance is ashwagandha (Withania somnifera). Ashwagandha has been documented as a rasayana (tonic) drug. The Rasayana acts both by providing resistance to the body to restrain or withstand the strength, severity or progression of a disease and also by improving physical strength of the body to prevent the manifestation of a disease. These rasayana herbs are common in practice as immunomodulator, antiviral and protectives.

Ashwagandha is a small or medium undershrub, 30-150 cm height, erect, grayish, branching perennial, with strong disagreeable odour like horse's urine. Almost the complete plant is covered and surrounded with very short, small, fine, branched hairs and silver-grey in colour. The stems of ashwagandha are brownish dark colour and erect, sometimes leaves are absent or less on lower part of stem. The leaves are in an alternate manner, simple, possessing margins are slightly waved, narrowed into the 5-20 mm long petioles, normally broadly ovate or oblong, 29-80 mm long and 21-50 mm broad. The roots of Ashwagandha are fleshy when dry, they are straight, cylindrical, tapering down, gradually unbranched of about 10-17.5 cm long and 6-12 mm diameter in thick. The main roots are brownish outer and creamy interior and bear fiber similar secondary roots having acrid taste and bitter. Ashwagandha roots are astringent, bitter, acrid, somniferous, thermogenic, stimulant, aphrodisiac, diuretic, deobstruent and tonic. They are useful in leucoderma, constipation, insomnia, lumbar pain, nervous disorders, asthma, cardiac disorders, psoriasis, consumption, ulcers, carbuncles, scabies, marasmus, and senile debility. Leaves are bitter and recommended in fever, painful swellings, inflammation of eye, syphilitic sores, hemorrhoids, tumors, tuberculous glands. Seeds are reported to be diuretic, hypnotic and are employed to coagulate milk.

This plant grows wildly in all drier parts of subtropical India. It occurs in Madhya Pradesh, Uttar Pradesh, Punjab plains and northwestern parts of India like Gujarat and Rajasthan. It is also found in Congo, South Africa, Egypt, Morocco, Jordan, Pakistan and Afghanistan.

In Ayurveda ashwagandha is used in many dosage forms like lepa (paste applied on affected area), kwath (decoction made boiling herb with Water), taila (medicated oils), dhooma (fumigation), ghrit (medicated ghee), churan (powder), kalka (paste/poultice), as agad (antidote).

### SUMMARY OF THE INVENTION

It is an object of this invention to provide a new and improved extract composition of ashwagandha roots which contains the full spectrum of active principles to provide maximum intended biological effect for the consumer.

It is a further object of the present invention to provide a process for the preparation of ashwagandha root extract fraction which is high yielding without using any organic solvent media for extraction.

In one general aspect, the present invention provides fortified extract composition of ashwagandha roots which contains the full spectrum of active principles present in the roots.

In an embodiment of the above aspect, the fortified extract is free of any organic solvent residue. Alternatively, the root extract is an aqueous root extract.

In another embodiment of the above aspect, is further fortified with milk or with biproducts of milk like whey, milk solids, curd etc.

In another general aspect, the present invention provides a process for the preparation of a fortified extract of ashwagandha roots, wherein the process comprises the following steps:
1. Making a decoction of ashwagandha roots with water in 1:16 weight ratio.
2. Soaking about 50 grams of coarse powder of ashwagandha roots (#30 pass) with 50 ml of Step 1 decoction.
3. Drying it naturally under shade or in hot air oven at 60-70° C.
4. Making a decoction of ashwagandha roots with water (1:10 weight ratio)
5. Soaking Step 3 powder of ashwagandha root with 50 ml of Step 4 decoction.
6. Drying it naturally under shade or in hot air oven at 60-70° C.
7. Making a decoction of ashwagandha root with water (1:8 weight ratio)
8. Soaking Step 6 powder of ashwagandha root with 50 ml of Step 7 decoction.
9. Drying it naturally under shade or in hot air oven at 60-70° C.
10. Making a decoction of ashwagandha root with water (1:6 weight ratio)
11. Soaking Step 9 powder of Ashwagandha with 50 ml of Step 10 decoction.
12. Drying it naturally under shade or in hot air oven at 60-70° C.
13. Making Mastu, as described in Bhav Prakash Nighantu of Bav Mishra or [symbol] —Sütrasthäna of Vagbhatta.
14. Soaking Step 12 powder of Ashwagandha with 50 ml Step 13 Mastu.
15. Drying it naturally under shade or in hot air oven at 60-70° C.
16. Soaking Step 15 powder of Ashwagandha with 50 ml of Step 14 Mastu.
17. Drying it naturally under shade/in Hot air oven at 60-70° C.
18. Soaking Step 17 powder of Ashwagandha with 50 ml of Step 14 Mastu.
19. Drying it naturally under shade/in Hot air oven at 60-70° C.

In an embodiment of the process, the process steps of treating dried powder of ashwagandha with Mastu is optional.

In yet another embodiment of the process, Mastu can be replaced by milk or by-products of milk except fats like ghee and vegetable oils.

### DETAILED DESCRIPTION OF THE INVENTION

The root extract of ashwagandha is a complex mixture of a large number of phytochemicals including phenolic compounds and flavonoids. The pharmacological effects of the roots of ashwagandha are attributed to withanolides, the naturally occurring steroids containing a lactone with a side chain of nine carbons, generally attached to C-17. Variation of lactone moiety is found in various classes of withanolides. Biologically active compounds usually occur in low concentration in plants. A most favorable or desirable extraction technique would be that which is able to obtain extracts with high yield and with minimal changes to the functional properties of the extract. Several studies have reported variations in the biological activities of extracts prepared using different extraction techniques. Therefore, it is necessary to select the suitable extraction method as well as solvent based on sample matrix properties, chemical properties of the analytes, matrix-analyte interaction, efficiency and desired properties.

The present invention provides for the potentiation of complete spectrum of naturally present active principles in the Ashwagandha. The root extract is prepared by aqueous extraction and no other organic chemical solvent media is used to extract active withanolides., To potentiate further the obtained whole spectrum ashwagandha root extract, it is treated with milk or with by-products of milk like whey, milk solids, curd etc.

The invention also provides a standardized process to obtain the potentiated whole spectrum ashwagandha complex. The process comprises of the steps:

### The Process:

1. To make a decoction of ashwagandha roots with water (in 1:16 ratio).
2. Soak 50 gm coarse powder of ashwagandha root (#30 pass) with 50 ml of step 1 decoction.
3. Dry it naturally under shade/in hot air oven at 60-70° C.
4. Water and ashwagandha root ratio can be varied to get the desired TDS's decoction.
5. Make a decoction of ashwagandha roots with water (in 1:10 ratio).
6. Soak Step 3 powder of ashwagandha roots with 50 ml of step 5 decoction.
7. Dry it naturally under shade/in hot air oven at 60-70° C.
8. Make a decoction of ashwagandha roots with water (in 1:8 ratio).
9. Soak Step 6 powder of ashwagandha root with 50 ml of step 8 decoction.
10. Dry it naturally under shade/in hot air oven at 60-70° C.
11. Make a decoction of ashwagandha roots with water (in 1:6 ratio).
12. Soak Step 9 powder of ashwagandha roots with 50 ml of step 11 decoction.
13. Dry it naturally under shade/in hot air oven at 60-70° C.
14. Make Mastu, as described in Bhav Prakash Nighantu of Bav Mishra or [symbol] -Sütrasthäna of Vagbhatta.
15. Soak Step 12 powder of Ashwagandha with 50 ml of step 14 Mastu.
16. Dry it naturally under shade/in hot air oven at 60-70° C.
17. Soak Step 16 powder of ashwagandha root with 50 ml of step 14 Mastu.
18. Dry it naturally under shade/in hot air oven at 60-70° C.
19. Soak Step 18 powder of Ashwagandha with 50 ml of step 14 Mastu.
20. Dry it naturally under shade/in ht air oven at 60-70° C.

The order of steps delineated above can be varied as well as multiplied. Also, in place of Mastu, milk or by-products of milk such as whey, milk solids, curd etc. except fat, ghee and vegetable oil can also be used.

### EXAMPLES OF PROCEDURE

### Example 1

| S. No. | Item/Parameter | Particulars | |
|---|---|---|---|
| 1 | Ashwagandha Roots | 50 | g |
| 2 | Water | 1000 | g |
| 3 | Heating Temperature | 100° | C. |
| 4 | Heating Time | 30 | minutes |
| 5 | Ashwagandha Roots (Whole) | 100 | g |
| 6 | Soaking Duration | 120 | min |
| 7 | Whey water | 20 | ml |
| 8 | Soaking Duration with Whey | 120 | min |

### Example 2

| S. No. | Item/Parameter | Particulars | |
|---|---|---|---|
| 1 | Ashwagandha Roots | 100 | g |
| 2 | Water | 1000 | g |
| 3 | Heating Temperature | 100° | C. |
| 4 | Heating Time | 45 | min |
| 5 | Ashwagandha Roots (Whole) | 100 | g |
| 6 | Soaking Duration | 180 | min |
| 7 | Whey water | 30 | ml |
| 8 | Soaking Duration with Whey | 100 | min |

### Example 3

| S. No. | Item/Parameter | Particulars | |
|---|---|---|---|
| 1 | Ashwagandha Roots | 10 | g |
| 2 | Water | 100 | g |
| 3 | Heating Temperature | 100° | C. |
| 4 | Heating Time | 15 | min |
| 5 | Ashwagandha Roots (Whole) | 10 | g |
| 6 | Soaking Duration | 180 | min |
| 7 | Whey water | 40 | ml |
| 8 | Soaking Duration with Whey | 180 | min |

### Example 4

| S. No. | Item/Parameter | Particulars | |
|---|---|---|---|
| 1 | Ashwagandha Roots | 100 | Kg |
| 2 | Water | 16K | Lt |
| 3 | Heating Temperature | 100° | C. |
| 4 | Heating Time | 300 | min |
| 5 | Ashwagandha Roots (Whole) | 1000 | Kg |
| 6 | Soaking Duration | 600 | min |
| 7 | Whey water | 20 | Lt |
| 8 | Soaking Duration with Whey | 1800 | min |

### Example 5

| S. No. | Item/Parameter | Particulars | |
|---|---|---|---|
| 1 | Ashwagandha Roots | 5000 | g |
| 2 | Water | 35 | Lt |
| 3 | Heating Temperature | 100° | C. |
| 4 | Heating Time | 360 | min |
| 5 | Ashwagandha Roots (Whole) | 10 | Kg |
| 6 | Soaking Duration | 1200 | min |
| 7 | Boiled Double toned Milk | 2500 | ml |
| 8 | Soaking Duration with Whey | 500 | min |

### Example 6

| S. No. | Item/Parameter | Particulars | |
|---|---|---|---|
| 1 | Ashwagandha Roots | 50 | g |
| 2 | Water | 2000 | ml |
| 3 | Heating Temperature | 100° | C. |
| 4 | Heating Time | 30 | min |
| 5 | Ashwagandha Roots (Whole) | 100 | g |
| 6 | Soaking Duration | 120 | min |
| 7 | Boiled Toned Milk | 30 | ml |
| 8 | Soaking Duration with Whey | 120 | min |

### Example 7

| S. No. | Item/Parameter | Particulars | |
|---|---|---|---|
| 1 | Ashwagandha Roots | 300 | g |
| 2 | Water | 5 | Lt |
| 3 | Heating Temperature | 100° | C. |
| 4 | Heating Time | 120 | min |
| 5 | Ashwagandha Roots (Whole) | 300 | g |
| 6 | Soaking Duration | 180 | min |
| 7 | Curdled milk water | 200 | ml |
| 8 | Soaking Duration with Whey | 180 | min |

### Example 8

| S. No. | Item/Parameter | Particulars | |
|---|---|---|---|
| 1 | Ashwagandha Roots | 4500 | g |
| 2 | Water | 15 | Lt |
| 3 | Heating Temperature | 100° | C. |
| 4 | Heating Time | 300 | min |
| 5 | Ashwagandha Roots (Whole) | 4500 | g |
| 6 | Soaking Duration | 540 | min |
| 7 | Curd water | 1000 | ml |
| 8 | Soaking Duration with Whey | 180 | min |

Process benefits observed:
1. Water Soluble extractive Increased.
2. Alcohol Soluble extractive reduced.

### Example 9

The compositions of the invention were tested for their in-vivo activity in healthy human subjects. A comparative study of safety and efficacy of a full-spectrum ashwagandha root composition was carried out to measure the effects in reducing stress and anxiety in adult human of either sex between the composition of the invention and a third party commercial ashwagandha product. The study included 10 adults in each groups aged between 18 to 45 years and did not have any psychiatric disorders. The subjects were divided into two groups A and B with 10 individuals in each groups of either sex. A third group was also taken as comparator with 10 subjects of either sex. The doses selected were 375 mg for Group A; 600 mg for Group B and 600 mg of the third party commercial ashwagandha product. The dose was once daily as capsule after meal for each group. The duration of the study the study was 30 days. The follow up was on day 0, 12th and 30th day. The blood samples were taken on the morning of 0 day and on day 30. The responses of standard questionnaire were taken on day 0, day 12 and day 30.

The blood samples were analyzed for initial levels and final levels on day 30 for morning cortisol levels, testosterone-free and total, thyroid hormone profile, C-reactive protein (CRP) test, luteinizing hormone and human growth hormone. The percentage difference (increase 1 or decrease 1) in the levels compared between the initial and the final are presented in the Table-1 below:

TABLE 1
Blood levels of different parameters in comparison to a commercial product

| | | Group A - 375 mg | | Group B - 600 mg | | Commercial Product - 600 mg | |
|---|---|---|---|---|---|---|---|
| S. No. | Parameter | Female | Male | Female | Male | Female | Male |
| 1 | Number | 5 | 4 | 2 | 8 | 2 | 7 |
| 2 | Mean age | 25.8 | 29.7 | 40.5 | 32.87 | 29.5 | 30.5 |
| 3 | Human | 157%↑ | 400%↑ | 157.5%↑ | 197.7%↑ | 79.16%↑ | 296%↑ |
| | Growth | | | | | | |
| | Hormone | | | | | | |
| | (hGH) | | | | | | |
| 4 | Cortisol | 21%↓ | 35%↓ | 22.8%↓ | 10.94%↓ | | 23.37%↓ |
| 5 | Testosterone - | | 9.6%↑ | | 3.857%↑ | | IS^{#} (3%↓) |
| | Free | | | | | | |
| 6 | Testosterone - | | 24.9%↑ | | 10.70%↑ | | 3.2%↑ |
| | Total | | | | | | |
| 7 | C-Reactive | | | 58%↓ | 11.92%↓ | | 32.9%↓ |
| | Protein | | | | | | |
| | (CRP) | | | | | | |
| 8 | TSH | 11.14%↓ | 25.09%↓ | 13.12%↓ | 33.36%↓ | 18.28%↓ | 4.61%↓ |
| 9 | LH | | 26.5%↑ | | 5.3%↑ | | 0.79%↑ |

^{#}Insignificant result

It is clear from Table 1 that there is significant change in the level of the tested parameters. For example, the hGH showed a growth of 157% (female) and 400% (male) over the base value of Day 0. The difference for hGH parameter between the composition of the invention and the commercial product was substantial. Similar results were obtained for testosterone-free as well as testosterone total. There was significant difference for luteinizing hormone parameters as well.

We claim:

1. A fortified extract composition of ashwagandha roots comprising the full spectrum of active principles present in the roots.

2. The fortified extract as claimed in claim 1, wherein the fortified extract is free of any organic solvent residue.

3. The fortified extract as claimed in claim 1, wherein the root extract is an aqueous root extract.

4. The fortified extract as claimed in claim 1, wherein the fortified extract is further fortified with milk or with biproducts of milk like whey, milk solids, curd etc.

5. A process for preparation of the fortified extract as claimed in claim 1, which is high yielding wherein the process does not use any organic solvent media for extraction.

6. The process as claimed in claim 5, wherein the process comprises the following steps:
I. Making a decoction of ashwagandha roots with water in 1:16 weight ratio.
II. Soaking about 50 grams of coarse powder of ashwagandha roots (#30 pass) with 50 ml of Step 1 decoction.
III. Drying it naturally under shade or in hot air oven at 60-70° C.
IV. Making a decoction of ashwagandha roots with water (1:10 weight ratio)
V. Soaking Step 3 powder of ashwagandha root with 50 ml of Step 4 decoction.
VI. Drying it naturally under shade or in hot air oven at 60-70° C.
VII. Making a decoction of ashwagandha root with water (1:8 weight ratio)
VIII. Soaking Step 6 powder of ashwagandha root with 50 ml of Step 7 decoction.
IX. Drying it naturally under shade or in hot air oven at 60-70° C.
X. Making a decoction of ashwagandha root with water (1:6 weight ratio)
XI. Soaking Step 9 powder of Ashwagandha with 50 ml of Step 10 decoction.
XII. Drying it naturally under shade or in hot air oven at 60-70° C.
XIII. Making Mastu, as described in Bhav Prakash Nighantu of Bav Mishra or [symbol] -Sütrasthäna of Vagbhatta.
XIV. Soaking Step 12 powder of Ashwagandha with 50 ml Step 13 Mastu.
XV. Drying it naturally under shade or in hot air oven at 60-70° C.
XVI. Soaking Step 15 powder of Ashwagandha with 50 ml of Step 14 Mastu.
XVII. Drying it naturally under shade/in Hot air oven at 60-70° C.
XVIII. Soaking Step 17 powder of Ashwagandha with 50 ml of Step 14 Mastu.
XIX. Drying it naturally under shade/in Hot air oven at 60-70° C.

7. The process as claimed in claim 6, wherein the steps of treating dried powder of ashwagandha with Mastu is optional.

8. The process as claimed in claim 7, wherein Mastu is replaced by milk or by-products of milk except fats like ghee and vegetable oils.
